(19) United States
(12) Patent Application Publication
(10) Pub. No.: US 2026/0021317 A1
(43) Pub. Date: Jan. 22, 2026

(54) Systems and Methods for Neurostimulation Targeting using Temporospatial Connectivity

(71) Applicant: The Board of Trustees of the Leland Stanford Junior University, Stanford, CA (US)

(72) Inventors: Nolan R. Williams, Incline Village, NV (US); Anish Mitra, Stanford, CA (US)

(73) Assignee: The Board of Trustees of the Leland Stanford Junior University, Stanford, CA (US)

(21) Appl. No.: 18/995,121

(22) PCT Filed: Jul. 14, 2023
(86) PCT No.: PCT/US2023/070270

§ 371 Date: Jan. 15, 2025

Related U.S. Application Data
(60) Provisional application No. 63/368,606, filed on Jul. 15, 2022.
(60) Provisional application No. 63/382,079, filed on Nov. 2, 2022.

(51) Int. Cl. A61N 2/00; A61N 1/20; A61N 2/02; A61N 7/00

(52) U.S. Cl. CPC A61N 2/006; A61N 1/20; A61N 2/02; A61N 7/00; A61N 2007/0026

(57) ABSTRACT

Systems and methods for fabricating a metal core truss panel with seamlessly embedded features in accordance with embodiments of the invention are illustrated. One embodiment includes a method of treatment for major depressive disorder including obtaining functional imaging data of a patient's brain, identifying disordered lagged correlations between different brain regions using the functional imaging data, identifying a first region of a dorsolateral prefrontal cortex most strongly anti-correlated with an anterior cingulate cortex, identifying a second region of the dorsolateral prefrontal cortex abnormally receiving signals from the anterior cingulate cortex, generating a neurostimulation target as the overlap between the first region and the second region, and applying neurostimulation to the target using a neurostimulation device.

### CROSS-REFERENCE TO RELATED APPLICATIONS

The current application is a U.S. National Stage of PCT Patent Application No. PCT/US2023/070270 entitled “Systems and Methods for Neurostimulation Targeting using Temporospatial Connectivity” filed Jul. 14, 2023, which claims the benefit of and priority under 35 U.S.C. § 119(e) to U.S. Provisional Patent Application No. 63/368,606 entitled “Systems and Methods for Objective Diagnosis and Treatment of Major Depressive Disorder” filed Jul. 15, 2022, and to U.S. Provisional Patent Application No. 63/382,079 entitled “Systems and Methods for Neurostimulation Targeting using Temporospatial Connectivity” filed Nov. 2, 2022. The disclosures of PCT Patent Application No. PCT/US2023/070270 and U.S. Provisional Patent Application Nos. 63/368,606 and 63/382,079 are hereby incorporated by reference in their entireties for all purposes.

### FIELD OF THE INVENTION

The present invention generally relates to the empirical identification of certain types of depression, generation of personalized neurostimulation targets based on temporospatial analysis of brain activation to treat said types of depression, and confirmation of effective treatment.

### BACKGROUND

Neurostimulation (also referred to as “neuromodulation”) is the practice of stimulating, either directly or indirectly, neurons in a patient. Neurostimulation can be achieved using both invasive and non-invasive modalities. Invasive forms of neurostimulation typically involve the implantation of neurostimulators into a patient's brain or body in order to directly stimulate neurons using electricity. For example, depending on the patient's needs, deep brain stimulation systems, epidural cortical stimulation systems, intracalvarial cortical stimulation systems, and subdural cortical stimulation systems can all be used as invasive neurostimulation options. Non-invasive forms of neurostimulation typically used for brain stimulation include transcranial magnetic stimulation (TMS), transcranial focused ultrasound (tFUS), and transcranial direct current stimulation (tDCS).

A connectome is a comprehensive map of neural connections in the brain. Connectomics is a science which acknowledges that structure and function of the human brain are linked. Different structures in the brain have different functionalities. Structures in the brain are also more closely linked to some structures than others. Functional connectivity refers to functionally integrated relationships between spatially separated brain regions. A number of networks which are typically shared amongst all humans are known such as (but not limited to) the default mode network, the salience network, the attention network, any many more.

Brain activity can be non-invasively measured using any of a number of functional neuroimaging techniques including (but not limited to), positron emission tomography (PET), functional magnetic resonance imaging (fMRI), functional near-infrared spectroscopy (fNIRS), and functional ultrasound imaging (fUS).

### SUMMARY OF THE INVENTION

Systems and methods for neurostimulation targeting using spatiotemporal connectivity in accordance with embodiments of the invention are illustrated. One embodiment includes a method of treatment for major depressive disorder including obtaining functional imaging data of a patient's brain, identifying disordered lagged correlations between different brain regions using the functional imaging data, identifying a first region of a dorsolateral prefrontal cortex most strongly anti-correlated with an anterior cingulate cortex, identifying a second region of the dorsolateral prefrontal cortex abnormally receiving signals from the anterior cingulate cortex, generating a neurostimulation target as the overlap between the first region and the second region, and applying neurostimulation to the target using a neurostimulation device.

In another embodiment, the neurostimulation is provided via a modality selected from the group consisting of: transcranial magnetic stimulation, transcranial direct current stimulation, transcranial focused ultrasound, and direct stimulation via implanted electrode.

In a further embodiment, the method includes confirming efficacy of treatment by obtaining a post-treatment functional imaging data of the patient's brain, and confirming reversion of the disordered lagged correlations.

In still another embodiment, the neurostimulation is accelerated theta burst stimulation.

In a still further embodiment, identifying disordered lagged correlations includes labeling voxels in the functional brain imaging data that make up the dorsolateral prefrontal cortex and the anterior cingulate cortex in the functional brain imaging data using a brain atlas, generating a whole-brain lagged correlation map using the voxels labelled as making up the anterior cingulate cortex as a reference region, where the whole-brain lagged correlation map includes a peak correlation magnitude and a peak temporal delay, computing a lag for all pairs of voxels containing grey matter, and assembling the computed lags into a time-delay matrix.

In yet another embodiment, the method includes computing the mean delay over all columns of the time-delay matrix to produce a lag projection map.

In a yet further embodiment, a neurostimulation targeting system for the treatment for major depressive disorder includes a processor, and a memory, the memory containing a target generation application which configures the processor to: obtain functional imaging data of a patient's brain, identify disordered lagged correlations between different brain regions using the functional imaging data, identify a first region of a dorsolateral prefrontal cortex most strongly anti-correlated with an anterior cingulate cortex, identify a second region of the dorsolateral prefrontal cortex abnormally receiving signals from the anterior cingulate cortex, generate a neurostimulation target as the overlap between the first region and the second region; and transmit the target to a neurostimulation device, where the neurostimulation device is configured to apply neurostimulation to the target.

In another additional embodiment, the neurostimulation device is selected from the group consisting of: a transcranial magnetic stimulation device, a transcranial direct current stimulation device, transcranial focused ultrasound device, and an implantable electrode.

In a further additional embodiment, the target generation application further directs the processor to: obtain a post-treatment functional imaging data of the patient's brain, confirm reversion of the disordered lagged correlations, and provide an indicator that the treatment was effective based on the confirmation.

In another embodiment again, the neurostimulation is accelerated theta burst stimulation.

In a further embodiment again, to identify disordered lagged correlations, the target generation application further configures the processor to: label voxels in the functional brain imaging data that make up the dorsolateral prefrontal cortex and the anterior cingulate cortex in the functional brain imaging data using a brain atlas, generate a whole-brain lagged correlation map using the voxels labelled as making up the anterior cingulate cortex as a reference region, where the whole-brain lagged correlation map includes a peak correlation magnitude and a peak temporal delay, compute a lag for all pairs of voxels containing grey matter, and assemble the computed lags into a time-delay matrix.

In still yet another embodiment, the target generation application further configures the processor to compute the mean delay over all columns of the time-delay matrix to produce a lag projection map.

In a still yet further embodiment, a neurostimulation device for the treatment for major depressive disorder includes a neurostimulator, a processor, and a memory, the memory containing a target generation application which configures the processor to: obtain functional imaging data of a patient's brain, identify disordered lagged correlations between different brain regions using the functional imaging data, identify a first region of a dorsolateral prefrontal cortex most strongly anti-correlated with an anterior cingulate cortex, identify a second region of the dorsolateral prefrontal cortex abnormally receiving signals from the anterior cingulate cortex, generate a neurostimulation target as the overlap between the first region and the second region, and transmit the target to the neurostimulation device, where the neurostimulator is configured to apply neurostimulation to the target.

In still another additional embodiment, the neurostimulator is a transcranial magnetic stimulation coil.

In a still further additional embodiment, the neurostimulator is a transcranial focused ultrasound transducer.

In still another embodiment again, the neurostimulator is a transcranial direct current stimulation electrode.

In a still further embodiment again, the target generation application further directs the processor to: obtain a post-treatment functional imaging data of the patient's brain, confirm reversion of the disordered lagged correlations, and provide an indicator that the treatment was effective based on the confirmation.

In yet another additional embodiment, the neurostimulation is accelerated theta burst stimulation.

In a yet further additional embodiment, to identify disordered lagged correlations, the target generation application further configures the processor to: label voxels in the functional brain imaging data that make up the dorsolateral prefrontal cortex and the anterior cingulate cortex in the functional brain imaging data using a brain atlas, generate a whole-brain lagged correlation map using the voxels labelled as making up the anterior cingulate cortex as a reference region, where the whole-brain lagged correlation map includes a peak correlation magnitude and a peak temporal delay, compute a lag for all pairs of voxels containing grey matter, and assemble the computed lags into a time-delay matrix.

In yet another embodiment again, the target generation application further configures the processor to compute the mean delay over all columns of the time-delay matrix to produce a lag projection map.

In a yet further embodiment again, the accelerated theta burst stimulation is applied in accordance with the Stanford Accelerated Intelligent Neurostimulation Therapy protocol.

In another additional embodiment again, a process for objectively diagnosing major depressive disorder includes obtaining a resting state functional magnetic imaging scan of a patient's brain describing a time series of brain activation, determining an ordering of brain structure activation based on the scan within a default mode network, a salience network, and a cognitive control network, and providing a likelihood of major depressive disorder when the ordering of brain structure activation is first temporo-parietal junction, second dorsal anterior cingulate, third anterior insula, then fourth dorsolateral prefrontal cortex.

In a further additional embodiment again, the disordered lagged correlations are an activation of the following brain structures in order: first temporo-parietal junction, second dorsal anterior cingulate, third anterior insula, then fourth dorsolateral prefrontal cortex.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a system diagram for a neurostimulation targeting system in accordance with an embodiment of the invention.

FIG. 2 is a block diagram for a target generator in accordance with an embodiment of the invention.

FIG. 3 is a flow chart for a target generation process in accordance with an embodiment of the invention.

FIG. 4 is a flow chart for a target generation process for MDD in accordance with an embodiment of the invention.

FIG. 5A-D is a set of charts illustrating computation of functional lag and a target in accordance with an embodiment of the invention.

FIG. 6 is a series of brain images showing example temporal changes in activation over time in response to stimulation at targets generated in accordance with an embodiment of the invention.

FIG. 7 is a set of charts that illustrate changes in reported depression with respect to temporal shifts in activation due to stimulation at targets generated in accordance with an embodiment of the invention.

FIG. 8 illustrates the difference in ACC latency between high responders and low responders to TMS treatment at targets generated in accordance with an embodiment of the invention.

FIG. 9 illustrates the difference in activation times between healthy brains and MDD brains in accordance with an embodiment of the invention.

### DETAILED DESCRIPTION

Depression is a debilitating mental condition that occurs with relatively high frequency in the population. Depression is the result of pathological brain circuitry. Multiple different brain circuitry abnormalities can give rise to depression. Studies have suggested that major depressive disorder (MDD) is likely caused by aberrant communication patterns in brain-wide networks. However, a mechanism-based biomarker for MDD has remained elusive. Traditionally, most studies have focused on either tonic activity levels in a given brain region or interactions between brain regions on the basis of zero-lag correlation magnitude (“functional connectivity”). The limitation of this conventional view of connectivity is that there is no assignment of signaling directionality. Thus, a circumstance in which brain region A signals to brain region B can appear identical to a circumstance in which the direction of communication is reversed, even though the neurobiological implications of communication direction may be profound.

Systems and methods described herein instead take a spatiotemporal view of connectivity, where lag times in activation between different correlated brain structures are measured in order to derive directionality of activation. For example, a particular type of depression is left dorsolateral prefrontal cortex (L-DLPFC)—anterior cingulate cortex (ACC) based major depressive disorder (MDD), where the depression arises from dysregulation of at least these two brain regions. In particular activation irregularly flows from the ACC to the DLPFC (i.e. ACC activation leads correlated DLPFC activation). Many other mental conditions may arise from similar irregular activation patterns Systems and methods described herein utilize the temporal activation arrangement of various structures within the brain to diagnose MDD. These pathological temporal arrangements provide objective biomarkers for diagnoses of many forms of MDD.

Furthermore, the pathological temporal arrangements can be used to generate a personalized neurostimulation target that is designed to push the pathological temporal arrangement into a healthy temporal arrangement. Using protocols described herein, MDD can be treated in patients by providing brain stimulation at their respective personalized target. Verification of treatment efficacy can be objectively determined by confirming the shift to healthy temporal arrangement post-stimulation. While the below focuses particularly on L-DLPFC—ACC based MDD, as can be readily appreciated, different pathological brain circuities may be associated with other classes of depression, or other neurological conditions. As such, similar approaches can be used with respect to different brain structures relevant to said other neurological conditions without departing from the scope or spirit of the invention. Neurostimulation targeting systems are discussed in further detail below.

### Neurostimulation Targeting Systems

Neurostimulation targeting systems obtain functional brain imaging data and use it to generate personalized neurostimulation targets. In many embodiments, neurostimulation targeting systems identify whether or not a given patient has a neurological condition which can be identified via spatiotemporal connectivity analysis, and/or whether or not they are likely to be a responder to neurostimulation. In various embodiments, neurostimulation targeting systems deliver neurostimulation at generated targets using neurostimulators.

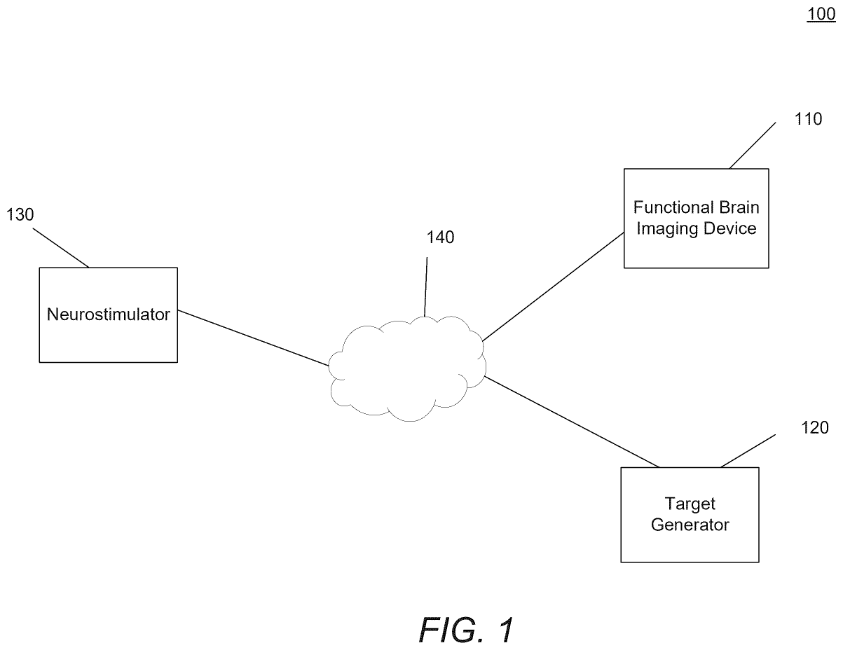

Turning now to FIG. 1, a neurostimulation targeting system in accordance with an embodiment of the invention is illustrated. System 100 includes a functional brain imaging device 110. Functional brain imaging devices are capable of imaging a patient's brain and recording a time-series of activation at different locations in the patient's brain. In numerous embodiments, the functional brain imaging device an fMRI machine, but any number of different functional brain imaging modalities can be used as appropriate to the requirements of specific applications of embodiments of the invention.

System 100 further includes a target generator 120. Target generators receive functional brain imaging data and generate neurostimulation targets based on spatiotemporal activation patterns within the functional brain imaging data. In many embodiments, the target generator identifies whether or not a patient has a neurological condition marked by abnormal spatiotemporal activation patterns. Target generators can determine whether or not an individual is likely to be a responder to neurostimulation. In various embodiments, conditions that are not marked by abnormal spatiotemporal activation patterns are unlikely to respond to neurostimulation. However particular abnormal spatiotemporal activation patterns may also indicate a non-responsive patient.

System 100 additionally includes a neurostimulator 130. Neurostimulators can be used to deliver stimulation of a given modality to targets generated by the target generator. In many embodiments, the neurostimulator is a TMS device, however the neurostimulator can use any number of different stimulation modalities including (but not limited to) tFUS and tDCS. In various embodiments, the neurostimulator is an invasive neurostimulator implanted such that electrical stimulation is directly or indirectly delivered to the generated target.

The functional brain imaging device, target generator, and the neurostimulator communicate over network 140. Networks can be made up of multiple networks or direct connections that are wired or wireless. In various embodiments, components of the system are not directly networked and instead data is passed between components via a physically transferred machine-readable medium. Further, as can readily be appreciated, any number of different architectures can be used including those that have multiple neurostimulators, operate using different modalities, or incorporate multiple components into the same hardware device (e.g. neurostimulator+target generator). In various embodiments, target generators operate on their own without the need for direct connection with neurostimulators and/or functional brain imaging devices.

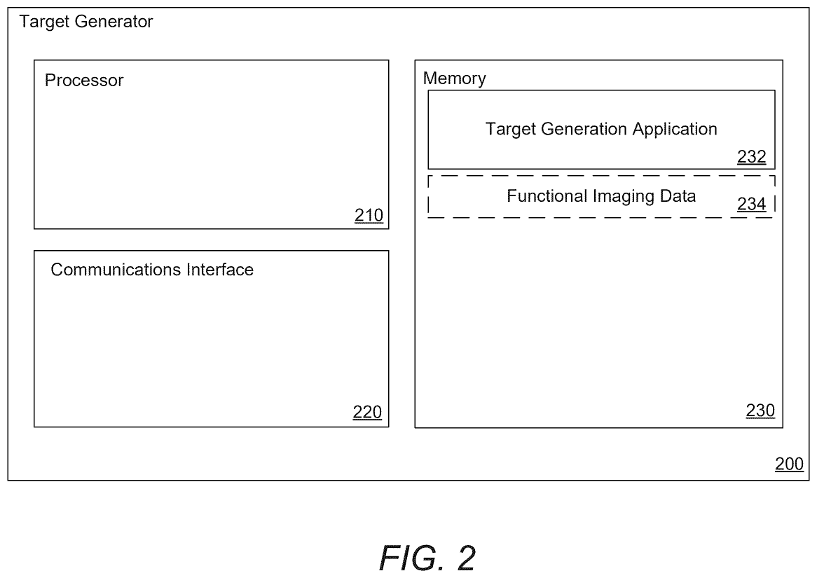

Turning now to FIG. 2, a target generator in accordance with an embodiment of the invention is illustrated. Target generator 200 has a processor 210. Processors can be a central processing unit (CPU), a graphics processing unit (GPU), an application-specific integrated circuit (ASIC), field-programable gate array (FPGA), and/or any other logic circuity as appropriate to the requirements of specific applications of embodiments of the invention. In various embodiments, target generators have more than one processor.

The target generator 200 further includes an input/output (I/O) interface 220. I/O interfaces can communicate via wired and/or wireless modalities to other devices such as (but not limited to) neurostimulators, functional brain imaging devices, displays, and/or any other electronic device as appropriate to the requirements of specific applications of embodiments of the invention.

Target generator 200 additionally contains a memory 230. Memory 230 can be made of volatile memory, nonvolatile memory, and/or any combination thereof. Memory 230 contains a target generation application 232 and is capable of storing functional imaging data 234. In many embodiments, target generation applications configure the processor to use received functional imaging data of a patient's brain to generate a personalized neurostimulation target using processes described herein. In various embodiments, target generation applications can configure the processor to determine if a patient will be a responder to neurostimulation treatment. In a variety of embodiments, the target generation application can configure the processor to use the neurostimulator to deliver stimulation at the generated target. While a particular architecture for target generators is shown, any number of different computing architectures can be used without departing from the scope or spirit of the invention. Processes for generating targets are discussed in further detail below.

### Generating Neurostimulation Targets

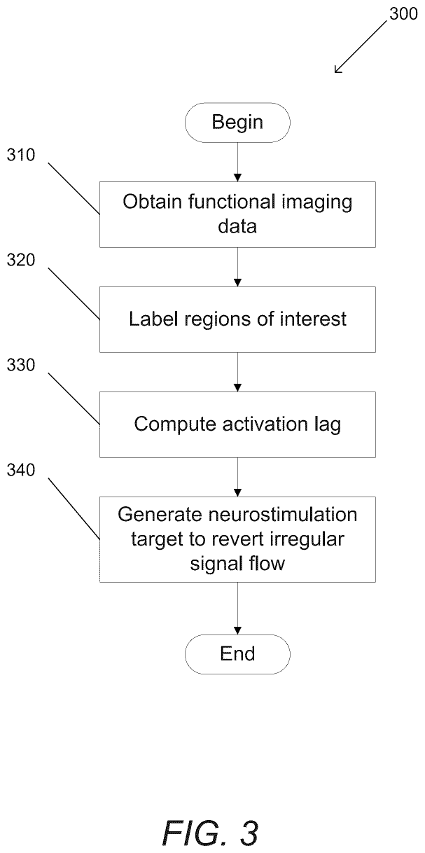

Neurostimulation targets are generated based on activation time in correlated brain structures such that stimulation at the target is intended to reverse the direction of activation in order to treat a neurological condition. Turning now to FIG. 3, a flow chart for generating neurostimulation targets based on brain activation times. Process 300 includes obtaining (310) functional imaging data of a patient's brain. In many embodiments, the functional imaging data is fMRI data containing blood oxygen level dependent (BOLD) signals for different voxels representing different brain regions. However, similar BOLD signals can be obtained using any of a variety of different modalities, and further similar temporal measurement of brain activation can be used as appropriate to the requirements of specific applications of embodiments of the invention.

Regions of interest are labeled such that each voxel is assigned to a particular brain structure. In many embodiments, labeling is achieved using a brain atlas. Based on activation over time between correlated regions of interest, activation lag is computed. In many embodiments, the activation lag imputes a direction of signal flow within the brain. A neurostimulation target can be generated (340) from within the region of interests that will reverse the irregular signal flow. As can be readily appreciated, the particular regions that are selected for analysis are dependent upon the condition to be treated.

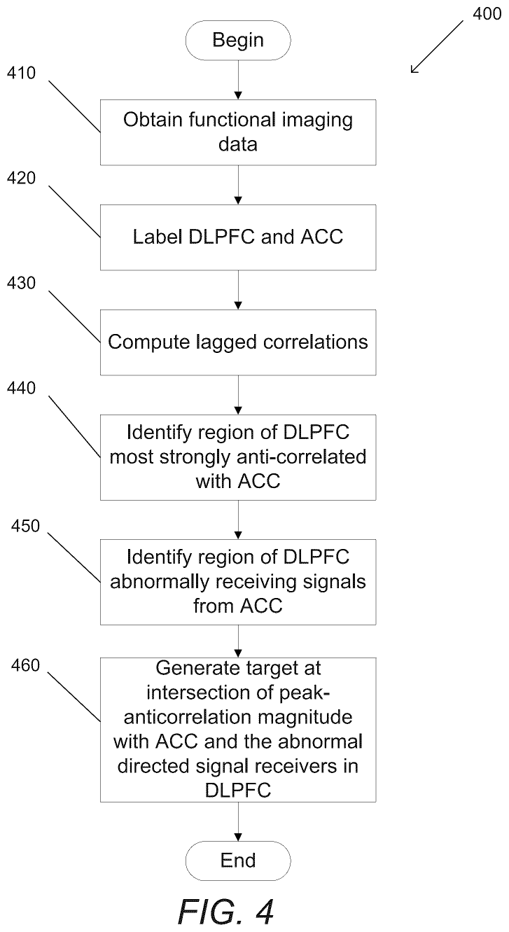

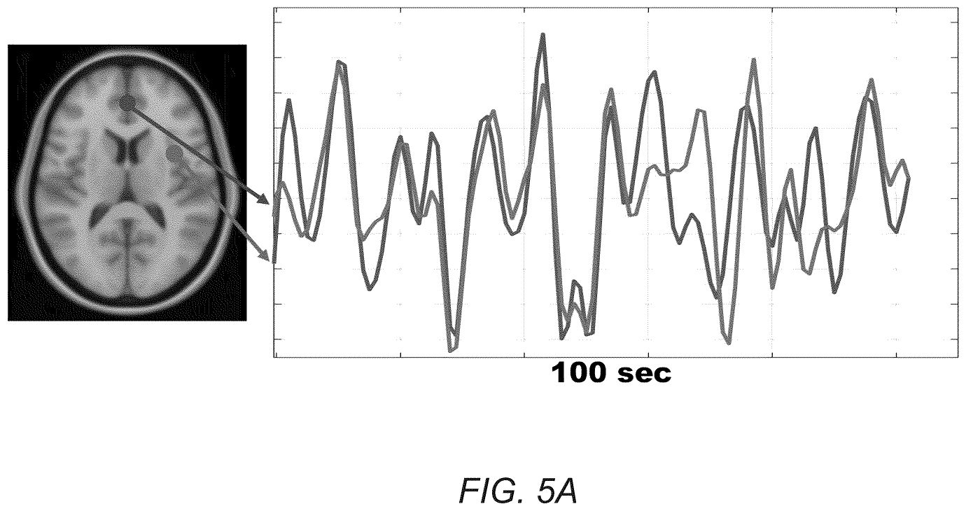

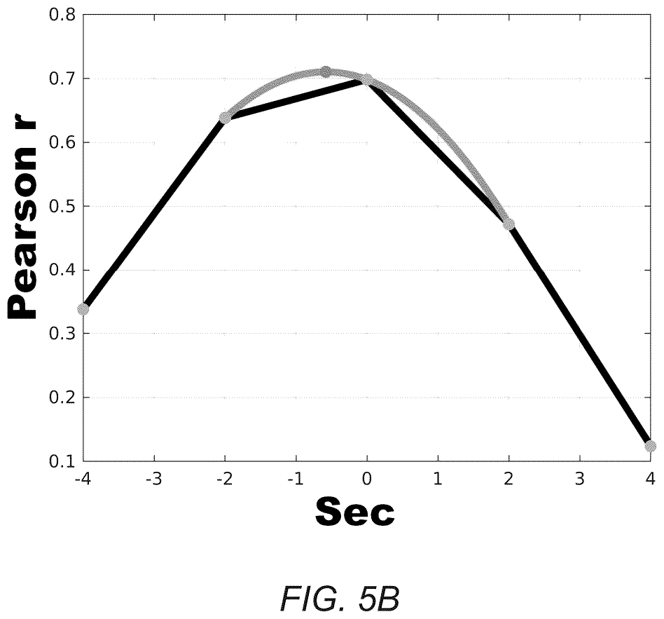

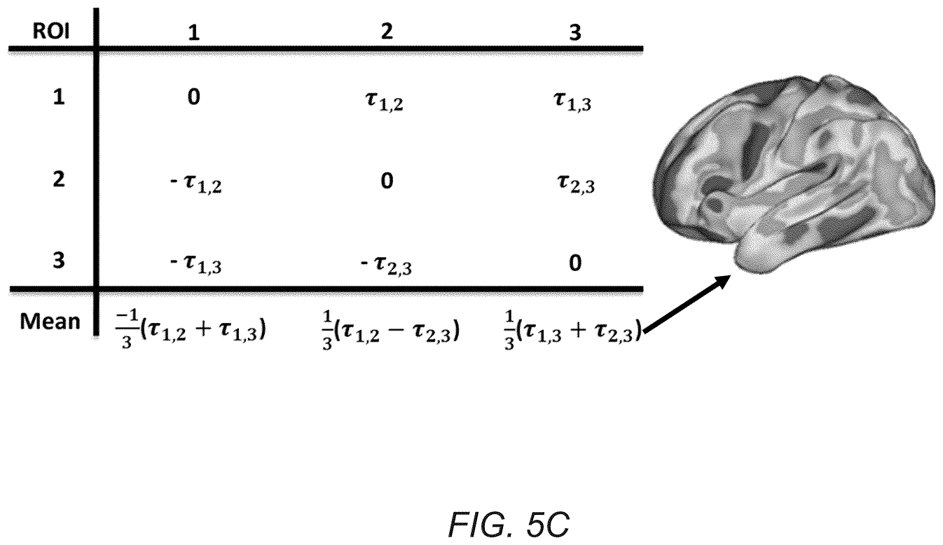

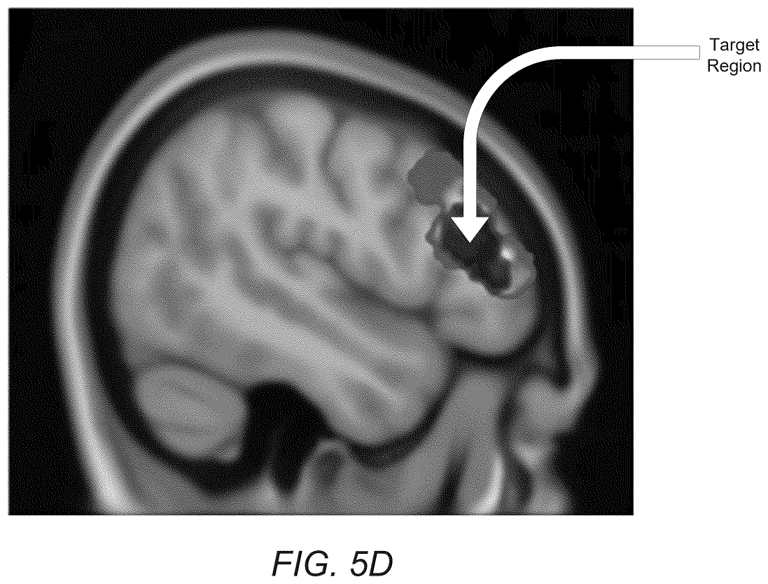

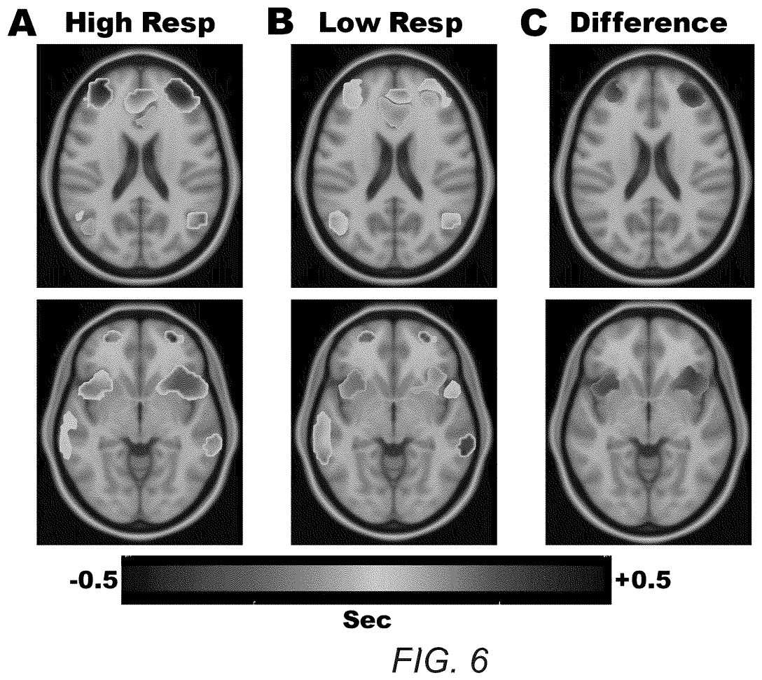

Turning now to FIG. 4, a similar process for generating a neurostimulation target to treat MDD in accordance with an embodiment of the invention is illustrated. Process 300 includes obtaining (410) functional imaging data. In many embodiments, the functional imaging data is a resting state fMRI (rs-fMRI). Within the functional imaging data, the dorsolateral prefrontal cortex (DLPFC) and the anterior cingulate cortex (ACC) are labeled (420). In many embodiments, the DLPFC and ACC are identified using segmentation of structural MRI scans, and/or any other structural scan. Using the anatomical cingulate area as a reference region, lagged correlations in the BOLD signal are computed (430) to generate a whole-brain lagged correlation map. An example BOLD signal for the DLPFC and the ACC are illustrated in FIG. 5A. The lagged correlation map includes two main parameters: peak correlation magnitude, and peak temporal delay.

In many embodiments, to calculate lagged correlations, first lags for all pairs of voxels in gray matter in the functional imaging data are computed. An example cross-correlation function for the time series in FIG. 5A is illustrated in FIG. 5B. In the illustrated embodiment, yellow markers and the black line depict empirically measured cross correlation. The green curve depicts a parabolic interpolation about the peak, with the orange marker at the interpolated peak showing a temporal offset of approximately 0.6 sec.

These results are assembled into time-delay (TD) matrices, which have dimensions voxels×voxels and entries in units of seconds. TD matrices represent the lag between all pairs of voxels in gray matter. The mean over all columns of the TD matrix is computed to yield a lag projection map. Lag projection maps topographically represent the mean lag between each voxel and the rest of the brain. The TD matrix format and an example lag projection map are illustrated in FIG. 5C.

The region of the DLPFC most strongly anticorrelated with the ACC is identified (440). In many embodiments, a threshold-based clustering on peak correlation magnitude can be applied to isolate the most strongly anticorrelated region after accounting for signaling delay. The region of the DLPFC which abnormally receives signals from the ACC is identified (450), i.e. the region of the DLPFC which lags the ACC.

The overlap between the two identified DLPFC regions represents the intersection of the peak anti-correlation magnitude with the ACC and the abnormal directed signal receivers in the DLPFC. When this overlapping region is stimulated via neurostimulation, the signal direction is reversed, and the patient experiences alleviation of their MDD symptoms. An example target as the overlap of the two identified regions is illustrated in FIG. 5D. In many embodiments, the patient is treated via neurostimulation of the target. In a variety of embodiments, the neurostimulation is accelerated theta burst stimulation as described in U.S. Pat. No. 10,595,735 titled “Systems and Methods for Personalized Clinical Applications of Accelerated Theta Burst Stimulation”, granted Mar. 24, 2020, the disclosure of which is hereby incorporated by reference in its entirety. Accelerated theta burst stimulation is also referred to as Stanford Accelerated Intelligent Neuromodulation Therapy, “SAINT”, or “SNT”.

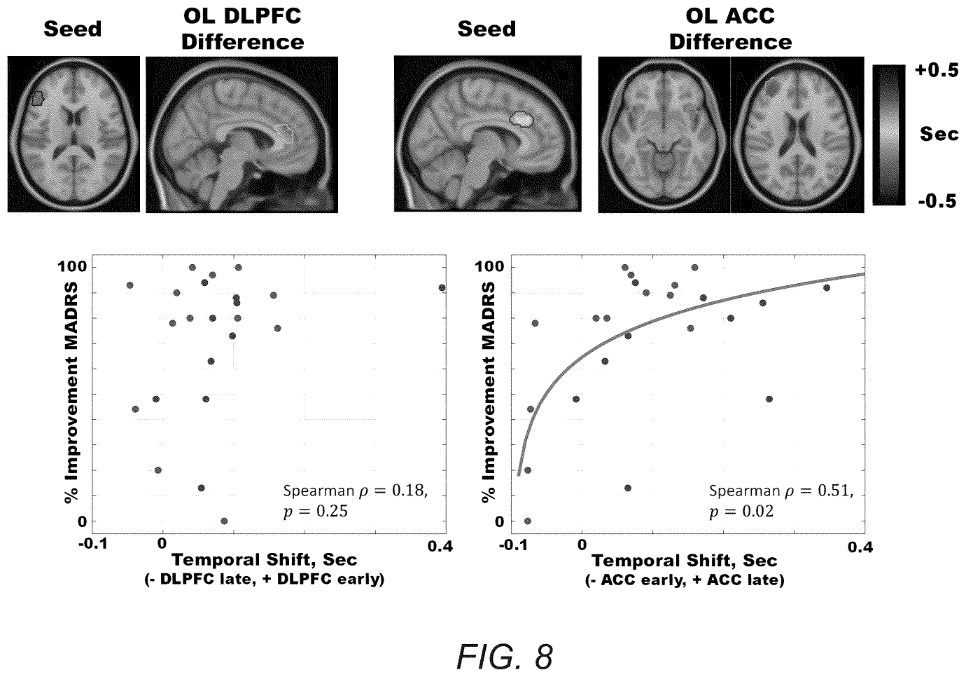

As noted above, the specific regions may be different for different conditions, however similar processing steps can be applied to identify targets for other conditions as appropriate to the requirements of specific applications of embodiments of the invention. Further, in many embodiments, whether or not a patient is likely to respond to a generated target can be determined using the brain activation patterns identified above. FIG. 8 shows baseline differences in ACC latency structure between high responders and low responders to TMS treatment. If similar latency patterns are present in a given patient, it may suggest that they are less likely to respond, or may respond to a lesser degree than would otherwise be expected. Treatment verification is discussed further below.

### Empirical Treatment Verification

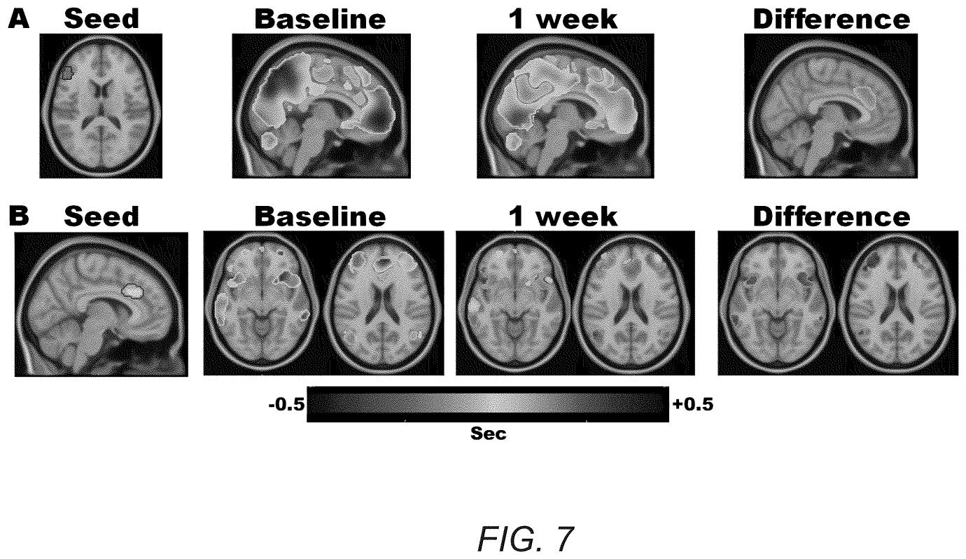

While how a patient feels is critical and in many cases paramount, empirical validation of a treatment can provide comfort to both the patient and the medical professionals. Similar to as how described above, lag can be recomputed based on functional brain imaging data obtained post treatment in order to see the change in activation direction. FIG. 7 illustrates changes in activation with respect to different seed regions from baseline, to one-week post stimulation. As can be seen, there is a significant difference in activation time that confirms the treatment worked. In the illustrated example, the top row is the seed-based lag map for the DLPFC cluster showing that the AAC shifts later with respect to the DLPFC, and the bottom row is the seed-based lag map for the ACC cluster showing that a series of regions, including the DLPFC, anterior insula, and posterior parietal cortices shift earlier with respect to the ACC.

FIG. 8 shows changes in seed-based latency vs changes in Montgomery- Asberg Depression Rating Scale (MADRS) scores, where blue dots are open label trial patients, and red dots were given sham-active stimulation. As can be seen, temporal shifting induced by stimulation reduced the reported depression.

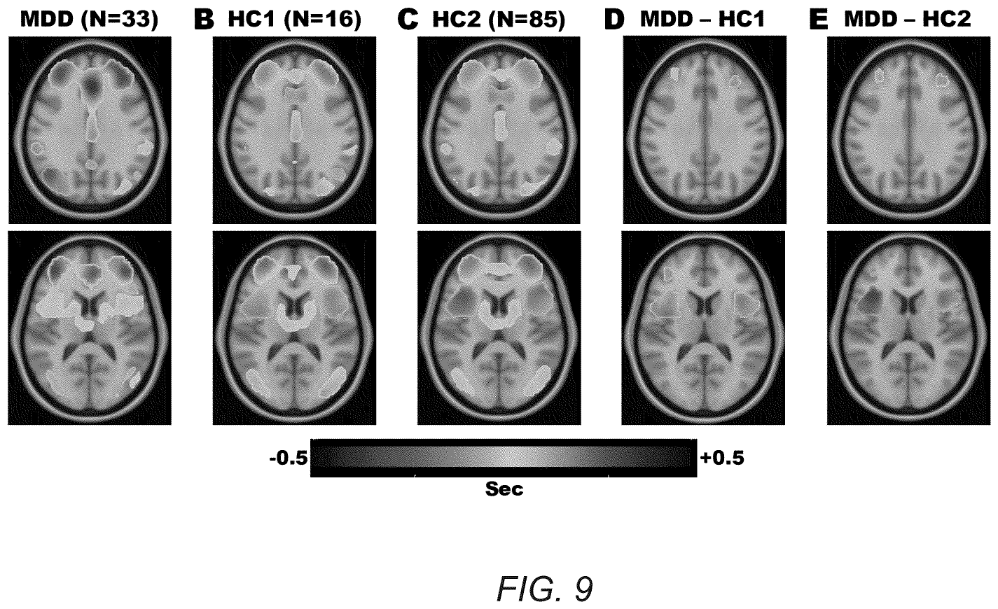

To provide further proof of efficacy of the described systems and methods, FIG. 9 illustrates ACC latency structure at baseline in MDD patients and two groups of healthy controls (HC1 & HC2). As can be seen, the lag problem in MDD patients persists across populations, and can be remediated as described herein in order to treat patients with MDD.

Although systems and methods for identifying certain types of MMD, generating targets for treatment, and confirming treatment efficacy are discussed above, many different architectures and methods can be implemented in accordance with many different embodiments of the invention which utilize spatiotemporal connectivity in a similar manner. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method of treatment for major depressive disorder, comprising:
obtaining functional imaging data of a patient's brain;
identifying disordered lagged correlations between different brain regions using the functional imaging data;
identifying a first region of a dorsolateral prefrontal cortex most strongly anti- correlated with an anterior cingulate cortex;
identifying a second region of the dorsolateral prefrontal cortex abnormally receiving signals from the anterior cingulate cortex;
generating a neurostimulation target as the overlap between the first region and the second region; and
applying neurostimulation to the target using a neurostimulation device.

2. The method of treatment of claim 1, wherein the neurostimulation is provided via a modality selected from the group consisting of: transcranial magnetic stimulation, transcranial direct current stimulation, transcranial focused ultrasound, and direct stimulation via implanted electrode.

3. The method of treatment of claim 1, further comprising confirming efficacy of treatment by:
obtaining a post-treatment functional imaging data of the patient's brain; and
confirming reversion of the disordered lagged correlations.

4. The method of treatment of claim 1, wherein the neurostimulation is accelerated theta burst stimulation.

5. The method of treatment of claim 1, wherein identifying disordered lagged correlations comprises:
labeling voxels in the functional brain imaging data that make up the dorsolateral prefrontal cortex and the anterior cingulate cortex in the functional brain imaging data using a brain atlas;
generating a whole-brain lagged correlation map using the voxels labelled as making up the anterior cingulate cortex as a reference region, where the whole-brain lagged correlation map comprises a peak correlation magnitude and a peak temporal delay;
computing a lag for all pairs of voxels containing grey matter; and
assembling the computed lags into a time-delay matrix.

6. The method of treatment of claim 5, further comprising computing the mean delay over all columns of the time-delay matrix to produce a lag projection map.

7. A neurostimulation targeting system for the treatment for major depressive disorder, comprising:
a processor; and
a memory, the memory containing a target generation application which configures the processor to:
obtain functional imaging data of a patient's brain;
identify disordered lagged correlations between different brain regions using the functional imaging data;
identify a first region of a dorsolateral prefrontal cortex most strongly anti- correlated with an anterior cingulate cortex;
identify a second region of the dorsolateral prefrontal cortex abnormally receiving signals from the anterior cingulate cortex;
generate a neurostimulation target as the overlap between the first region and the second region; and
transmit the target to a neurostimulation device, where the neurostimulation device is configured to apply neurostimulation to the target.

8. The system of claim 7, wherein the neurostimulation device is selected from the group consisting of: a transcranial magnetic stimulation device, a transcranial direct current stimulation device; transcranial focused ultrasound device; and an implantable electrode.

9. The system of claim 7, where the target generation application further directs the processor to:
obtain a post-treatment functional imaging data of the patient's brain;
confirm reversion of the disordered lagged correlations; and
provide an indicator that the treatment was effective based on the confirmation.

10. The system of claim 7, wherein the neurostimulation is accelerated theta burst stimulation.

11. The system of claim 7, wherein to identify disordered lagged correlations, the target generation application further configures the processor to:
label voxels in the functional brain imaging data that make up the dorsolateral prefrontal cortex and the anterior cingulate cortex in the functional brain imaging data using a brain atlas;
generate a whole-brain lagged correlation map using the voxels labelled as making up the anterior cingulate cortex as a reference region, where the whole-brain lagged correlation map comprises a peak correlation magnitude and a peak temporal delay;
compute a lag for all pairs of voxels containing grey matter; and
assemble the computed lags into a time-delay matrix.

12. The system of claim 11, wherein the target generation application further configures the processor to compute the mean delay over all columns of the time-delay matrix to produce a lag projection map.

13. A neurostimulation device for the treatment for major depressive disorder, comprising:
a neurostimulator;
a processor; and
a memory, the memory containing a target generation application which configures the processor to:
obtain functional imaging data of a patient's brain;
identify disordered lagged correlations between different brain regions using the functional imaging data;
identify a first region of a dorsolateral prefrontal cortex most strongly anti- correlated with an anterior cingulate cortex;
identify a second region of the dorsolateral prefrontal cortex abnormally receiving signals from the anterior cingulate cortex;
generate a neurostimulation target as the overlap between the first region and the second region; and
transmit the target to the neurostimulation device;
where the neurostimulator is configured to apply neurostimulation to the target.

14. The neurostimulation device of claim 13, wherein the neurostimulator is a transcranial magnetic stimulation coil.

15. The neurostimulation device of claim 13, wherein the neurostimulator is a transcranial focused ultrasound transducer.

16. The neurostimulation device of claim 13, wherein the neurostimulator is a transcranial direct current stimulation electrode.

17. The neurostimulation device of claim 13, where the target generation application further directs the processor to:
obtain a post-treatment functional imaging data of the patient's brain;
confirm reversion of the disordered lagged correlations; and
provide an indicator that the treatment was effective based on the confirmation.

18. The neurostimulation device of claim 13, wherein the neurostimulation is accelerated theta burst stimulation.

19. The neurostimulation device of claim 13, wherein to identify disordered lagged correlations, the target generation application further configures the processor to:
label voxels in the functional brain imaging data that make up the dorsolateral prefrontal cortex and the anterior cingulate cortex in the functional brain imaging data using a brain atlas;
generate a whole-brain lagged correlation map using the voxels labelled as making up the anterior cingulate cortex as a reference region, where the whole-brain lagged correlation map comprises a peak correlation magnitude and a peak temporal delay;
compute a lag for all pairs of voxels containing grey matter; and
assemble the computed lags into a time-delay matrix.

20. The neurostimulation device of claim 19, wherein the target generation application further configures the processor to compute the mean delay over all columns of the time-delay matrix to produce a lag projection map.
